(19) United States
(12) Patent Application Publication
(10) Pub. No.: US 2026/0232582 A1
(43) Pub. Date: Aug. 13, 2026

(54) PHARMACEUTICAL COMPOSITION OF ENSIFENTRINE WITH PHARMACEUTICALLY ACCEPTABLE PROPELLANTS

(71) Applicant: CIPLA LIMITED, Mumbai (IN)

(72) Inventors: Kiran Rote, Mumbai (IN); Pradeep Bhadauria, Weston, FL (US); Priyanka Bajpayee, Mumbai (IN); Arijit Das, Mumbai (IN); Ramanaiah Chennuru, Andhra Pradesh (IN)

(73) Assignee: CIPLA LIMITED, Mumbai, MH (IN)

(21) Appl. No.: 19/469,369

(22) PCT Filed: Mar. 28, 2024
(86) PCT No.: PCT/IN2024/050331

§ 371 Date: Sep. 25, 2025

(30) Foreign Application Priority Data
Mar. 28, 2023 (IN) 202321022560

(51) Int. Cl. A61K 9/00; A61K 31/519; A61K 47/06

(52) U.S. Cl. CPC A61K 9/008; A61K 31/519; A61K 47/06

(57) ABSTRACT

Provided is a composition of inhalation either by oral or nasal route comprising ensifentrine or pharmaceutically acceptable salt or ester thereof, pharmaceutically acceptable propellant and a pharmaceutically acceptable excipient. More preferably, provided is a composition of inhalation either by oral or nasal route comprising ensifentrine or pharmaceutically acceptable salt or ester thereof, pharmaceutically acceptable propellant having GWP less than 200 and a pharmaceutically acceptable excipient.

### PRIORITY DETAILS

This application claims the benefit of Indian Provisional Application No. 202321022560 filed on Mar. 28, 2023, which is hereby incorporated by reference in its entirety.

### TECHNICAL FIELD OF AN INVENTION

Provided is a composition of inhalation either by oral or nasal route comprising ensifentrine or pharmaceutically acceptable salt or ester thereof, pharmaceutically acceptable propellant and a pharmaceutically acceptable excipient. More preferably, provided is a composition of inhalation either by oral or nasal route comprising ensifentrine or pharmaceutically acceptable salt or ester thereof, pharmaceutically acceptable propellant having GWP less than 200 and a pharmaceutically acceptable excipient.

### BACKGROUND

Ensifentrine (Also known as RPL554) combines bronchodilator and anti-inflammatory properties in one compound and has the potential to be an effective treatment for respiratory disorders such as chronic obstructive pulmonary disease (COPD), asthma, and cystic fibrosis. Ensifentrine is a first-in-class, inhaled, dual inhibitor of the phosphodiesterase 3 (PDE-3) and phosphodiesterase 4 (PDE-4) enzymes. This selective dual inhibition enables it to combine bronchodilator and anti-inflammatory properties in one compound, differentiating it from existing drug classes used to treat COPD, including corticosteroids, beta 2-agonists and anti-muscarinics. Ensifentrine has been well tolerated in clinical trials involving more than 2,200 subjects. It is designed to maximize its effectiveness and reduce adverse events through high dual selectivity of phosphodiesterase 3 and 4 (PDE-3 and PDE-4) over other enzymes and receptors to minimize off-target effects; and direct delivery to the lungs by inhalation to maximize pulmonary exposure to ensifentrine while minimizing systemic distribution and potential adverse events. Verona Pharma evaluated nebulized ensifentrine in the phase 3 clinical program ENHANCE (Ensifentrine as a Novel inhaled Nebulized COPD therapy) for COPD maintenance treatment. Ensifentrine met the primary endpoint in both ENHANCE-1 and ENHANCE-2 trials demonstrating statistically significant and clinically meaningful improvements in lung function. In addition, ensifentrine significantly reduced the rate and risk of COPD exacerbations in pooled analysis from ENHANCE-1 and ENHANCE-2. There are also reports on positive Phase 2 results with dry powder inhalation (DPI) and pressurized metered dose inhalation (pMDI) formulations of ensifentrine demonstrating significant improvements in lung function in COPD patients.

Administration of ensifentrine by nebulizer is known as disclosed in WO 2016/042313A1. However, it is often desirable to administer respiratory drugs using the pressurized metered dose inhalation (pMDI). pMDI formulations comprising salts of ensifentrine are disclosed in WO 2015/173551A1 and WO 2020/074894A1. A pMDI formulation comprising a combination of ensifentrine with a second active agent is described in WO 2014/140648A1 and WO2021/171034A1. Certain formulations of ensifentrine have been disclosed in WO2024/033624A1, WO2024/033625A1, WO2024/033626A1 and WO2024/033627A1. Known pMDI formulation strategies can involve the use of a wide range of different propellants, excipients, co-solvents, and surfactants, and may take the form of suspensions, solutions or mixtures thereof. For instance, pMDI formulations may include one or more propellants such as alkanes (for instance propane, n-butane, isobutane, n-pentane, isopentane, neopentane), hydrofluoroalkanes (HFAs, for instance HFA-227ea, HFA-134a and HFA-152a), ethers (for instance dimethylether) and hydrofluoro-olefins (HFOs, for instance HFO-1234z (E) and HFO-1234yf). Co-solvents such as ethanol and water are commonly included in pMDI formulations. Excipients which are commonly included in pMDI formulations include antioxidants, preservatives, wetting agents, chelating agents, emulsifiers, flavorings, buffers, lubricants, suspending agents and tonicity adjusting agents. Each of the components included in pMDI can have several effects on formulation stability and efficacy which are highly dependent on the identity of the drug to be formulated. Such effects cannot reliably be predicted in advance. Appropriate formulation of the drug for use in a pMDI is critical to ensure that the formulation can be used successfully in clinical settings.

Increases in global temperature are already having a significant impact on our climate. The hydrofluorocarbon (HFC) propellants used today in pressurized metered-dose inhalers (pMDIs) have global warming potential (GWP) many times that of carbon dioxide. Their use, together with all other emissive uses of HFCs, is being phased down under the Montreal protocol. It is projected that the reduction in non-medical uses of propellants is likely to give rise to a 5-fold increase in their cost for pMDI uses and is likely to hit the western world in 2025 (Pritchard JN. The Climate is Changing for Metered-Dose Inhalers and Action is Needed. Drug Des Devel Ther. 2020; 14:3043-3055. Published 2020 Jul. 29. doi: 10.2147/DDDT.S262141). This may lead to a price increase in reliever medication that will make it unaffordable for the poorer communities in some markets. At the same time, opportunities to save money by developing new formulations using propellants with lower GWP, such as HFA-152a or HFO-1234ze (E) is a need of the hour because of their lower GWP. However, as new propellants, it is extremely likely that the regulators will expect long-term human safety data to be collected before granting market authorization. This will undoubtedly be a critical and given that neither low GWP propellant is widely available in pharmaceutical-grade material, noryet has a comprehensive inhalation safety data package, there is need to develop a stable formulation using such propellants. Disclosed herein are stable compositions of ensifentrine with propellants (HFA-152a or HFO-1234ze (E)) having low GWP.

### SUMMARY OF INVENTION

In one aspect of the invention, there is provided a composition of inhalation either by oral or nasal route comprising ensifentrine or salt or ester thereof, pharmaceutically acceptable green propellant having GWP less than 200.

In one aspect of the invention, there is provided a composition of inhalation either by oral or nasal route comprising ensifentrine or pharmaceutically acceptable salt or ester thereof, a combination of pharmaceutically acceptable green propellant having GWP less than 200 and GWP more than 200, and a pharmaceutically acceptable excipient.

In yet another aspect, provided is a pharmaceutical composition for treatment or prevention of respiratory disorder in a subject in need thereof, comprising ensifentrine or pharmaceutically acceptable salt or ester thereof, and a green propellant. In one embodiment, the composition further comprises of one or more of a pharmaceutically acceptable excipient. In another embodiment, the pharmaceutically acceptable excipient is selected from the group consisting of a co-solvent, a surfactant, a lubricant, a bulking agent, an acid, or a combination thereof. In another embodiment, the ensifentrine or pharmaceutically acceptable salt or ester thereof is present in a concentration from about 0.001% w/w to about 5% w/w.

In yet another aspect, provided is a pharmaceutical composition for treatment or prevention of respiratory disorder in a subject in need thereof, comprising ensifentrine or pharmaceutically acceptable salt or ester thereof, and a green propellant; wherein the propellant is selected from HFA-152a, HFO-1234ze (E) or a combination thereof. In one embodiment, the propellant HFA-152a is present in the concentration from about 1% to about 99% w/w of composition. In another embodiment, the propellant HFO-1234ze (E) is present in the concentration from about 1% to about 99% w/w of composition. In yet another embodiment, the propellant is the combination of HFA-152a and HFO-1234ze (E). In yet another aspect, provided is a pharmaceutical suspension composition for treatment or prevention of respiratory disorder in a subject in need thereof, comprising particles of ensifentrine or pharmaceutically acceptable salt or ester thereof, and a green propellant; and wherein the particles of ensifentrine have a particle size distribution with a Dv50 value of from about 1 μm to about 10 μm; wherein the composition is suitable for administration by oral or nasal inhalation; wherein the composition can be administered using a pressurized metered dose inhaler or a nebulizer.

In yet another aspect, provided is a pharmaceutical solution composition for treatment or prevention of respiratory disorder in a subject in need thereof, comprising about 0.01% to 2.5% w/w of ensifentrine or pharmaceutically acceptable salt or ester thereof, and HFA-152a or HFO-1234ze (E); wherein the ensifentrine is present in the form of extra fine particles having Dv50 of less than 1 μm.

In still another aspect provided is a process of preparing compositions as per preceding aspects.

### DETAILED DESCRIPTION

Ensifentrine (Also known as RPL554) combines bronchodilator and anti-inflammatory properties in one compound and has the potential to be an effective treatment for respiratory disorders such as COPD, asthma, and cystic fibrosis. Ensifentrine is a first-in-class, inhaled, dual inhibitor of the phosphodiesterase 3 (PDE-3) and phosphodiesterase 4 (PDE-4) enzymes. This selective dual inhibition enables it to combine bronchodilator and anti-inflammatory properties in one compound, differentiating it from existing drug classes used to treat COPD, including corticosteroids, beta2-agonists and anti-muscarinics. Ensifentrine has been well tolerated in clinical trials involving more than 2,200 subjects. It is designed to maximize its effectiveness and reduce adverse events through high dual selectivity of phosphodiesterases 3 and 4 (PDE-3 and PDE-4) over other enzymes and receptors to minimize off-target effects; and direct delivery to the lungs by inhalation to maximize pulmonary exposure to ensifentrine while minimizing systemic distribution and potential adverse events. Verona Pharma evaluated nebulized ensifentrine in the Phase 3 clinical program ENHANCE (Ensifentrine as a Novel inhaled Nebulized COPD therapy) for COPD maintenance treatment. Ensifentrine met the primary endpoint in both ENHANCE-1 and ENHANCE-2 trials demonstrating statistically significant and clinically meaningful improvements in lung function. In addition, ensifentrine significantly reduced the rate and risk of COPD exacerbations in pooled analysis from ENHANCE-1 and ENHANCE-2. There are also reports on positive Phase 2 results with DPI and pMDI formulations of ensifentrine demonstrating significant improvements in lung function in COPD patients.

In the first embodiment of the invention, disclosed is a composition for inhalation either by oral or nasal route comprising ensifentrine or pharmaceutically acceptable salt or ester thereof, a pharmaceutically acceptable green propellant having GWP less than 200.

In another embodiment, disclosed is a pharmaceutical composition for treatment or prevention of respiratory disorder in a subject in need thereof, comprising ensifentrine or pharmaceutically acceptable salt or ester thereof, and a green propellant.

In another embodiment, disclosed is a composition for inhalation comprising ensifentrine or pharmaceutically acceptable salt or ester thereof, a pharmaceutically acceptable green propellant having GWP less than 200, the propellant having GWP more than 200, and a pharmaceutically acceptable excipient.

In the yet another embodiment of the invention, disclosed is a composition for inhalation comprising ensifentrine or pharmaceutically acceptable salt or ester thereof, one or more pharmaceutically acceptable green propellant having GWP less than 200 and a pharmaceutically acceptable excipient.

Ensifentrine or pharmaceutically acceptable salt or ester thereof is present in a concentration from about 0.001% to 5% w/w of the composition, or from about 0.01% to 2.5% w/w of composition. In some embodiments, ensifentrine or pharmaceutically acceptable salt or ester thereof is present in about 0.001% w/w, or 0.01% w/w, or 0.1% w/w, or 0.5% w/w, or 0.8% w/w, or 1% w/w, or 1.5% w/w, or 2% w/w, or 2.5% w/w of the composition. Preferably, composition comprises about 0.56% w/w, or about 0.83% w/w of ensifentrine or pharmaceutically acceptable salt or ester thereof. Alternatively, the composition comprises about 5 mcg to 2000 mcg, or about 10 mcg to about 1000 mcg, or about 50 mcg to about 500 mcg, or about 150 mcg of ensifentrine or pharmaceutically acceptable salt or ester thereof. Alternatively, composition comprises about 250 mcg, or about 375 mcg of ensifentrine or pharmaceutically acceptable salt or ester thereof. The salts of ensifentrine are selected from but not limited to group comprising ethane-1,2-disulfonic acid, ethanesulfonic acid, methanesulfonic acid, benzenesulfonic acid, p-toluenesulfonic acid, hydrochloric acid, hydrobromic acid, phosphoric acid or sulfuric acid, or solvates thereof. In an alternate embodiment, ensifentrine is present as free base in the composition. The composition of the invention also comprises a pharmaceutically acceptable propellant. The propellant should also be capable of maintaining the drug in a homogeneous solution, in a stable suspension or in a stable dispersion for a sufficient time to permit reproducible delivery of the drug in use. When the drug is in suspension in the propellant, the density of the liquid propellant is desirably like that of the solid drug to avoid rapid sinking or floating of the drug particles in the liquid. Finally, the propellant should not possess a significant flammability risk to the patient in use. It should form a non-flammable or low flammability mixture when mixed with air in the respiratory tract. Dichlorodifluoromethane (R-12) possesses a suitable combination of properties and was for many years the most widely used pMDI propellant, often blended with trichlorofluoromethane (R-11). Due to international concern that fully and partially halogenated chlorofluorocarbons (CFCs), such as dichlorodifluoromethane and trichlorofluoromethane, were damaging the earth's protective ozone layer, many countries entered into an agreement, the Montreal Protocol, stipulating that their manufacture and use should be severely restricted and eventually phased out completely. Dichlorodifluoromethane and trichlorofluoromethane were phased out for refrigeration use in the 1990's but are still used in small quantities in the MDI sector because of an essential use exemption in the Montreal Protocol. 1,1,1,2-tetrafluoroethane (HFA-134a) was introduced as a replacement refrigerant and MDI propellant for R-12. 1,1,1,2,3,3,3-heptafluoropropane (HFA-227ea) was also introduced as a replacement propellant for dichlorotetrafluoroethane (R-114) in the MDI sector and is, sometimes used alone or blended with HFA-134a for this application. Although HFA-134a and HFA-227ea have low ozone depletion potentials (ODPs), they have global warming potentials (GWPs), 1430 and 3220 respectively, which are now considered to be too high by some regulatory bodies, especially for dispersive uses when they are released into the atmosphere. One industrial area that has received particular attention recently has been the automotive air-conditioning sector where the use of HFA-134a has come under regulatory control because of the European Mobile Air Conditioning Directive (2006/40/EC). Industry has developed several possible alternatives to HFA-134a in automotive air conditioning and other applications that have a low greenhouse warming potential (GWP) as well as a low ozone depletion potential (ODP). Many of these alternatives include hydrofluoropropenes, especially tetrafluoropropenes, such as 2,3,3,3-tetrafluoropropene (HFO-1234yf) and 1,3,3,3-tetrafluoropropene (HFO-1234ze (E)). Although the proposed alternatives to HFA-134a have a low GWP, the toxicological status of many of the components, such as certain of the fluoropropenes, is unclear and they are unlikely to be acceptable for use in the MDI sector for many years, if at all. Further, Koura has developed Zephex 152 (HFA-152a) is a new, sustainable medical propellant for use in pMDIs for treatment of respiratory disorders such as asthma and COPD. Zephex 152 minimizes the carbon footprint of pMDIs while safeguarding an invaluable therapeutic option for patients. This new propellant will reduce the carbon footprint of pMDI inhalers by up to 90%, down to a similar level as a dry powder inhaler. Therefore, taking from above, two propellants HFO-1234ze (E) and HFA-152a have been referred to as “Green Propellants” as their GWP is less than 200. The invention composition in some embodiment comprises one green propellant such HFO-1234ze (E) or HFA-152a, or combination of green propellant such as HFO-1234ze (E) and HFA-152a, or combination of any one green propellant with other non-green propellant (such as HFO-1234ze (E) and HFA-134a, or HFA-152a and HFA-134a, or HFO-1234ze (E) and HFA-227ea, or HFA-152a and HFA-227ea). The total concentration of propellant ranges from 10 to 90% by weight of the composition or 10 to 99% by weight of the composition or in preferred embodiments to q.s to 100%. Alternatively, if more than one propellant is present in the composition, the total concentration of propellant ranges from 10 to 90% by weight of the composition.

The invention composition further comprises a pharmaceutically acceptable excipient which is, but not limited to, surfactant or suspending agent (such as polyvinyl pyrrolidone, polysorbates, sorbitan esters of fatty acid, and oleic acid), co-solvent (such as ethanol, polyethylene glycol), lubricant (such as such as silica, talc, stearic acid, magnesium or calcium stearate), bulking agent (such as starches, arabic gums, gelatin, methylcellulose, carboxymethylcellulose) or organic or inorganic acids (such as HCl, sulphuric acid, phosphoric acid, citric acid, maleic acid, malic acid, boric acid, methane sulphonic acid, tartaric acid or oxalic acid), or combination of forgoing. In some embodiment, the pharmaceutically acceptable excipient is the combination of co-solvent and surfactant, or the combination of co-solvent and lubricant, or the combination of surfactant/suspending agent and lubricant, or the combination of co-solvent, surfactant, and lubricant, or the combination of co-solvent and acid.

The surfactant or suspending agent is present in a concentration ranging from 0.001 to 1% by weight of the composition. 0.001% to about 1% w/w of the composition. In another embodiment, the surfactant is polyvinylpyrrolidone (PVP) and is present in the concentration from about 0.001% to about 0.005% w/w, more preferably, in about 0.001% w/w of the composition. Various grades of polyvinylpyrrolidone can be used such as PVP K12, PVP K25 or PVP K30. Alternatively, any PVP grade ranging from K value of 12 to 30 can be used. In some embodiments, the surfactant is oleic acid, or orbitan trioleate or sorbitan mono-oleate and are present in the concentration from about 0.01% to about 0.5% w/w, more preferably in about 0.03% w/w of the composition.

The co-solvent is present in a concentration ranging from 0.1 to 20% by weight of the composition. In some embodiments, the co-solvent is ethanol and is present in about 2% w/w, or 5% w/w, or 10% w/w, or 15% w/w of the composition. In some embodiment, the co-solvent is polyethylene glycol (various grades from 100 to 5000 can be used) and is present in about 0.1% w/w, or about 0.5% w/w, or about 1% w/w, or about 2% w/w, or about 4% w/w, or about 6% w/w, or about 8% w/w, or about 10% w/w, or 15% w/w, or about 20% w/w of the composition.

The lubricant is present in a concentration ranging from 0.001 to 2% by weight of the composition. In another embodiment, the preferred concentration of lubricant is about 0.01% w/w, or about 0.05% w/w, or about 0.1% w/w, or about 0.5% w/w, or about 1% w/w, or about 1.5% w/w of the composition. The preferred concentration is about 0.1% w/w of the composition.

Organic or inorganic acid is present in a concentration ranging from 0.001 to 2% by weight of the composition. In another embodiment, the preferred concentration of organic or inorganic acid is about 0.01% w/w, or about 0.05% w/w, or about 0.1% w/w, or about 0.5% w/w, or about 1% w/w, or about 1.5% w/w of the composition. The preferred concentration is about 0.024% w/w of the composition.

Preferably, the composition of the invention is formulated for administration by inhalation. The pharmaceutical composition may be formulated as solution or suspension and administered using a pressurized metered dose inhaler or nebulizer. Preferably, the invention compositions are administered using the pressurized metered dose inhaler. In a suspension formulation, ensifentrine has particle size distribution with a Dv50 (median particle size by volume) value of from about 0.2 μm to about 5 μm. The particle size is measured using standard techniques such as laser diffraction. In some embodiments, when the formulation can be prepared as solution, the Dv50 of ensifentrine particles is less than 2 μm, preferably less than 1 μm. Such solutions are referred to as extra fine composition of ensifentrine.

In another embodiment, there is provided a composition of inhalation either by oral or nasal route comprising ensifentrine or pharmaceutically acceptable salt or ester thereof, a combination of pharmaceutically acceptable green propellant having GWP less than 200 and GWP more than 200, and a pharmaceutically acceptable excipient.

In one embodiment, disclosed is a pharmaceutical composition comprising ensifentrine or pharmaceutically acceptable salt or ester thereof, and a pharmaceutically acceptable propellants selected from HFA-152a, or HFO-1234ze (E), or HFA-134a, or HFA-227ea.

In another embodiment, disclosed is a pharmaceutical composition comprising ensifentrine or pharmaceutically acceptable salt or ester thereof, and a pharmaceutically acceptable propellant comprising combination of HFA-152a and HFA-134a.

In yet another embodiment, disclosed is a pharmaceutical composition comprising ensifentrine or pharmaceutically acceptable salt or ester thereof, and a pharmaceutically acceptable propellant comprising combination of HFA-152a and HFO-1234ze (E).

In yet another embodiment, disclosed is a pharmaceutical composition comprising ensifentrine or pharmaceutically acceptable salt or ester thereof, and a pharmaceutically acceptable propellant comprising combination of HFA-134a and HFO-1234ze (E).

In yet another embodiment, disclosed is a pharmaceutical composition for treatment or prevention of respiratory disorder in a subject in need thereof, comprising ensifentrine or pharmaceutically acceptable salt or ester thereof, and a green propellant, wherein the composition further comprises of one or more of a pharmaceutically acceptable excipient, and wherein the pharmaceutically acceptable excipient is selected from the group consisting of a co-solvent, a surfactant, a lubricant, a bulking agent, an acid, or a combination thereof.

In yet another embodiment, disclosed is a pharmaceutical composition for treatment or prevention of respiratory disorder in a subject in need thereof, comprising ensifentrine or pharmaceutically acceptable salt or ester thereof, and a green propellant; wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof; wherein the ensifentrine or pharmaceutically acceptable salt or ester thereof is selected from ethane-1,2-disulfonic acid, ethanesulfonic acid, methanesulfonic acid, benzenesulfonic acid, p-toluenesulfonic acid, hydrochloric acid, hydrobromic acid, phosphoric acid, sulfuric acid, or solvates thereof.

In yet another embodiment, disclosed is a pharmaceutical composition for treatment or prevention of respiratory disorder in a subject in need thereof, comprising about 0.001% w/w to about 5% w/w ensifentrine or pharmaceutically acceptable salt or ester thereof, and a green propellant; wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant, wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant HFA-152a; wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant is combination of HFA-152a and HFO-1234ze (E); wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E), or a combination thereof; wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof; wherein the propellant HFA-152a is present in the concentration from about 1% to about 99% w/w of composition; and wherein the propellant HFO-1234ze (E) is present in the concentration from about 1% to about 99% w/w of composition.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises of one or more of a pharmaceutically acceptable excipient; and wherein the pharmaceutically acceptable excipient is selected from the group consisting of a co-solvent, a surfactant, a lubricant, a bulking agent, an acid, or a combination thereof; wherein the propellant HFA-152a is present in the concentration from about 1% to about 99% w/w of composition; and wherein the propellant HFO-1234ze (E) is present in the concentration from about 1% to about 99% w/w of composition; wherein the co-solvent is selected from a group consisting of ethanol, glycerol, polyethylene glycol or combination thereof; wherein the co-solvent is present in the concentration from about 0.1% to about 20% w/w of composition.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises ethanol which is present in the concentration from about 0.1% to about 20% w/w of composition.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises surfactant selected from group consisting of oleic acid, sorbitan trioleate, sorbitan mono-oleate, polyvinylpyrrolidone, polysorbate or combinations thereof; and wherein surfactant is present in the concentration from about 0.001% to about 1% w/w of the composition.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises polyvinylpyrrolidone which is present in the concentration from about 0.001% to about 0.005% w/w.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises oleic acid which is present in the concentration from about 0.01% to about 0.5% w/w.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises sorbitan trioleate or sorbitan mono-oleate which is present in the concentration from about 0.001% to about 0.5% w/w of the composition.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises lubricant selected from a group consisting of silica talc, stearic acid, magnesium or calcium stearate or combinations thereof; and wherein the lubricant is present in the concentration from about 0.001% to about 2% w/w of composition.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises magnesium or calcium stearate; and is present in the concentration from about 0.001% to about 2% w/w of composition.

In yet another embodiment, disclosed is a pharmaceutical composition for treatment or prevention of respiratory disorder in a subject in need thereof, comprising about 0.56% w/w or about 0.83% w/w ensifentrine or pharmaceutically acceptable salt or ester thereof, and a green propellant selected from HFA-152a, HFO-1234ze (E) or a combination thereof; wherein the composition further comprises acid selected from group consisting of hydrochloric acid, sulphuric acid, phosphoric acid, citric acid, maleic acid, malic acid, boric acid, methane sulphonic acid, tartaric acid, oxalic acid, or combinations thereof; and wherein the acid is present in the concentration from about 0.001 to about 2% w/w of the composition.

In still another embodiment of the invention, disclosed is a pharmaceutical suspension composition for treatment or prevention of respiratory disorder in a subject in need thereof, comprising particles of ensifentrine or pharmaceutically acceptable salt or ester thereof, and a green propellant; and wherein the particles of ensifentrine have a particle size distribution with a Dv50 value of from about 1 μm to about 5 μm.

In still another embodiment of the invention, disclosed is a pharmaceutical suspension composition for treatment or prevention of respiratory disorder in a subject in need thereof, comprising particles of ensifentrine or pharmaceutically acceptable salt or ester thereof, and a green propellant; and wherein the particles of ensifentrine have a particle size distribution with a Dv50 value of from about 1 μm to about 5 μm; wherein the composition is suitable for administration by oral or nasal inhalation.

In still another embodiment of the invention, disclosed is a pharmaceutical suspension composition for treatment or prevention of respiratory disorder in a subject in need thereof, comprising particles of ensifentrine or pharmaceutically acceptable salt or ester thereof, and a green propellant; and wherein the particles of ensifentrine have a particle size distribution with a Dv50 value of from about 1 μm to about 5 μm; wherein the composition is suitable for administration by oral or nasal inhalation; and wherein the pharmaceutical composition can be administered using a pressurized metered dose inhaler or a nebulizer. Alternatively, the inventions can be prepared as a pharmaceutical solution composition for treatment or prevention of respiratory disorder in a subject in need thereof, comprising about 0.01% to 2.5% w/w of ensifentrine or pharmaceutically acceptable salt or ester thereof, and HFA-152a; wherein the ensifentrine is present in the form of extra fine particles having Dv50 of less than 1 μm.

In one embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea or combination thereof, and a surfactant or suspending agent. In another embodiment, the surfactant or suspending agent is polyvinyl pyrrolidone, oleic acid, sorbitan esters of fatty acid or combination thereof.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea or combination thereof, and polyvinyl pyrrolidone K25.

In yet another embodiment, disclosed is a pharmaceutical composition comprising ensifentrine or pharmaceutically acceptable salt or ester thereof, a pharmaceutically acceptable propellant HFA-152a, and polyvinyl pyrrolidone K25.

In yet another embodiment, disclosed is a pharmaceutical composition comprising ensifentrine or pharmaceutically acceptable salt or ester thereof, a pharmaceutically acceptable propellant HFO-1234ze (E), and polyvinyl pyrrolidone.

In yet another embodiment, disclosed is a pharmaceutical composition comprising ensifentrine or pharmaceutically acceptable salt or ester thereof, a pharmaceutically acceptable propellants comprising combination of HFA-152a and HFO-1234ze (E), and polyvinyl pyrrolidone.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, and sorbitan esters of fatty acids.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, a co-solvent, and a surfactant and/or suspending agent.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, ethanol and polyvinyl pyrrolidone.

In yet another embodiment, disclosed is a pharmaceutical composition comprising ensifentrine or pharmaceutically acceptable salt or ester thereof, a pharmaceutically acceptable propellant HFA-152a, ethanol and polyvinyl pyrrolidone.

In yet another embodiment, disclosed is a pharmaceutical composition comprising ensifentrine or pharmaceutically acceptable salt or ester thereof, a pharmaceutically acceptable propellant HFO-1234ze (E), ethanol and polyvinyl pyrrolidone.

In yet another embodiment, disclosed is a pharmaceutical composition comprising ensifentrine or pharmaceutically acceptable salt or ester thereof, a pharmaceutically acceptable propellant comprising combination of HFO-1234ze (E), and HFA-152a, ethanol, and polyvinyl pyrrolidone.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, ethanol, and oleic acid.

In yet another embodiment, disclosed is a pharmaceutical composition comprising ensifentrine or pharmaceutically acceptable salt or ester thereof, a pharmaceutically acceptable propellant HFA-152a, ethanol, and oleic acid.

In yet another embodiment, disclosed is a pharmaceutical composition comprising ensifentrine or pharmaceutically acceptable salt or ester thereof, a pharmaceutically acceptable propellant HFO-1234ze (E), ethanol, and oleic acid.

In yet another embodiment, disclosed is a pharmaceutical composition comprising ensifentrine or pharmaceutically acceptable salt or ester thereof, a pharmaceutically acceptable propellant comprising combination of HFA-152a, and HFO-1234ze (E), ethanol, and oleic acid.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, ethanol, and sorbitan esters of fatty acids.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, and a lubricant.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, and polyethylene glycol.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, a co-solvent and a lubricant.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, ethanol, and polyethylene glycol.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, a surfactant and/or suspending agent, and a lubricant. In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, polyvinyl pyrrolidone, and polyethylene glycol.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, a surfactant and/or suspending agent, a lubricant, and co-solvent.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, polyvinyl pyrrolidone, polyethylene glycol, and ethanol.

In yet another embodiment, disclosed is a pharmaceutical composition comprising ensifentrine or pharmaceutically acceptable salt or ester thereof, a pharmaceutically acceptable propellant selected from HFA-152a, or HFO-1234ze (E), or HFA-134a, or HFA-227ea, or combination thereof, and a lubricant.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, and magnesium stearate.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, and an organic or inorganic acid.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, and maleic acid.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, an organic or inorganic acid and a co-solvent.

In yet another embodiment, disclosed is a pharmaceutical composition comprising ensifentrine or pharmaceutically acceptable salt or ester thereof, a pharmaceutically acceptable propellants selected from HFA-152a, or HFO-1234ze (E), or HFA-134a, or HFA-227ea, or combination thereof, an organic or inorganic acid, and ethanol.

In still another embodiment, provided are the process of preparing a pharmaceutical composition having ensifentrine and propellants.

The invention compositions can be used for prevention and treatment of respiratory disorders such as asthma, chronic obstructive pulmonary disease, cystic fibrosis and other inflammatory respiratory disorders. The compositions can also be used for maintenance treatment of asthma and COPD.

The invention is described by way of the examples below and the scope of the invention is not limited by the boundaries of examples.

### EXAMPLES

### Example 1: Ensifentrine Compositions with Green Propellant

TABLE 1

| | 1A | 1B | 1C | 1D |
|---|---|---|---|---|
| Ingredient | (% w/w) | (% w/w) | (% w/w) | (% w/w) |
| Ensifentrine | 0.56 | 0.83 | 0.56 | 0.83 |
| Propellant HFA 152a | 99.44 | 99.17 | — | — |
| Propellant HFO- | — | — | 99.44 | 99.17 |
| 1234ze(E) | | | | |

*The 0.56% w/w and 0.83% w/w correspond to 250 mcg and 375 mcg of Ensifentrine.

Manufacturing Process:
Ensifentrine required for batch manufacturing was weighed.
Ensifentrine was homogenized with part quantity of propellant HFA-152a or HFO-1234ze (E) to obtain a suspension.
Homogenized suspension was transferred to a mixing vessel and mixing was carried out with the remaining quantity of propellant.
After completion of mixing, suspension was recirculated through the filling line and filled in pre-crimped canister.
After filling canisters were quarantined for 14 days and evaluated for the fine particle fraction and (FPM) and uniform delivery dose (UDD) test using know methods in art.

### Example 2: Fine Particle Fraction and (FPM) and Uniform Delivery Dose (UDD) of Ensifentrine Compositions with Green Propellant

Compositions 1A and 1B were subjected to FPM and DDU test conducted as per the known methods in the art. Below are the results of the test.

TABLE 2
Aerodynamic particle size distribution

| Strength | IP | S1 | S2 | S3 | S4 | S5 | S6 | S7 | MOC | MB | FPM | MMAD |
|---|---|---|---|---|---|---|---|---|---|---|---|---|
| 250 mcg | 106.6 | 3.0 | 6.4 | 22.1 | 53.5 | 25.1 | 5.4 | 1.0 | 0.5 | 223.5 | 98.1 | 3.0 |
| 375 mcg | 166.3 | 8.3 | 18.3 | 40.7 | 77.6 | 29.9 | 5.9 | 1.2 | 0.7 | 347.9 | 136.3 | 3.4 |

TABLE 3
Uniformity of delivered dose (in %)

| Strength | Initial - 1 | Initial - 2 | Initial - 3 | Middle - 1 | Middle - 2 | Middle - 3 | Middle - 4 | End - 1 | End - 2 | End - 3 | Mean |
|---|---|---|---|---|---|---|---|---|---|---|---|
| 250 mcg | 98 | 99 | 101 | 111 | 109 | 109 | 111 | 109 | 108 | 110 | 100 |
| 375 mcg | 89 | 92 | 90 | 102 | 99 | 102 | 100 | 111 | 107 | 108 | 100 |

The overall FPM and DDU performance of the compositions were better and with less the variability.

### Example 3: Ensifentrine Compositions

| Ingredients | | | | | | | | | | | |
|---|---|---|---|---|---|---|---|---|---|---|---|
| (% w/w) | 3A | 3B | 3C | 3D | 3E | 3F | 3G | 3H | 3I | 3J | 3K |
| Ensifentrine | 0.56 or 0.83 | | | | | | | | | | |
| PVP/PVP K25 | — | — | — | — | — | 0.001 | 0.001 | — | — | 0.001 | 0.001 |
| Ethanol | — | — | — | — | — | — | — | — | — | 2 | 2 |
| PEG1000/400 | — | — | — | — | 0.1 | — | — | — | — | — | — |
| Oleic Acid/ Sorbitan | — | — | — | — | — | — | — | — | — | — | — |
| Trioleate/ Sorbitan | | | | | | | | | | | |
| mono-oleate | | | | | | | | | | | |
| Mg. stearate | — | — | — | — | — | — | — | 0.1 | 0.1 | — | — |
| Mineral acid/ weak | — | — | — | — | — | — | — | — | — | — | — |
| acid | | | | | | | | | | | |
| Propellant HFA-134a | — | — | 10 | 10 | — | — | — | — | — | — | — |
| Propellant HFA-152a | 99.56 | — | 89.56 | — | 99.46 | 99.56 | — | 99.46 | — | 97.56 | — |
| Propellant HFO- | — | 99.66 | — | 89.66 | — | — | 99.66 | — | 99.56 | — | 97.66 |
| 1234ze(E) | | | | | | | | | | | |

% w/w

| Ingredients | | | | | | | | | | | |
|---|---|---|---|---|---|---|---|---|---|---|---|
| (% w/w) | 3L | 3M | 3N | 3O | 3P | 3Q | 3R | 3S | 3T | 3U | 3V |
| Ensifentrine | 0.56 or 0.83 | | | | | | | | | | |
| PVP/PVP K25 | 0.001 | 0.001 | — | — | — | — | — | — | — | — | — |
| Ethanol | 2 | 2 | 2 | 2 | 2 | 2 | — | 15 | 15 | 15 | 15 |
| PEG1000/400 | 0.1 | 0.1 | 0.1 | 0.1 | — | — | — | — | — | — | — |
| Oleic Acid/ Sorbitan | | | — | — | 0.03 | 0.03 | 0.03 | — | — | 0.03 | 0.03 |
| Trioleate/ Sorbitan | | | | | | | | | | | |
| mono-oleate | | | | | | | | | | | |
| Mg. stearate | | | — | — | — | — | — | — | — | — | — |
| Mineral acid/ weak | | | — | — | — | — | — | 0.024 | 0.024 | — | — |
| acid | | | | | | | | | | | |
| Propellant HFA-134a | | | — | — | — | — | — | — | — | — | — |
| Propellant HFA-152a | 97.46 | — | 97.46 | — | 99.43 | — | 99.53 | 84.54 | — | 84.53 | — |
| Propellant HFO- | — | 97.56 | — | 97.56 | — | 99.73 | — | — | 84.64 | — | 84.63 |
| 1234ze(E) | | | | | | | | | | | |

Manufacturing process for formulation containing Ensifentrine and propellant.
Weighed the required quantity of ensifentrine in a canister and crimped with the valve.
The canister was charged with the required quantity of the propellant.
The canister was sonicated for 1-90 min and quarantined the canister for stabilization.

Manufacturing process for formulation containing ensifentrine, surfactant, and propellant.
The surfactant was dissolved in the propellant.
The required quantity of ensifentrine was weighed in canister and crimped the canister with valve.
The canister was charged with the required quantity of mixture of surfactant and remaining propellant.
The canister was sonicated for 1-90 min and quarantined the canister for stabilization.

Manufacturing process for formulation containing ensifentrine, ethanol, Surfactant and Propellant
The surfactant was dissolved in ethanol and propellant.
The required quantity of ensifentrine was weighed in the canister and crimped the canister with valve.
The canister was charged with the required quantity of mixture of surfactant, ethanol, and propellant.
The canister was sonicated for 1-90 min and quarantined the canister for stabilization.

Manufacturing process for formulation containing ensifentrine, ethanol, acid, and propellant.
The acid was dissolved in ethanol.
The ensifentrine was dissolved in the above mixture.
The required quantity of the above mixture was weighed in canister and crimped the canister with valve.

The canister was charged with the required quantity of propellant.
The canister was sonicated for 1-90 min and quarantined the canister for stabilization.

Valve: Valve with metering chamber capacity from 50-63 mcl was used.

Canister: Plain or FCP coated canister with a capacity of 14-19 ml was used.

We claim:

1. A pharmaceutical composition for treatment or prevention of respiratory disorder in a subject in need thereof, comprising ensifentrine or pharmaceutically acceptable salt or ester thereof, and a green propellant.

2. (canceled)

3. The pharmaceutical composition of claim 1, wherein the composition further comprises one or more of a pharmaceutically acceptable excipient, preferably the one or more pharmaceutically acceptable excipient which is selected from the group consisting of: a co-solvent, a surfactant, a lubricant, a bulking agent, an acid, and a combination thereof.

4. The pharmaceutical composition of claim 1, wherein the ensifentrine or pharmaceutically acceptable salt or ester thereof is selected from ethane-1,2-disulfonic acid, ethanesulfonic acid, methanesulfonic acid, benzenesulfonic acid, p-toluenesulfonic acid, hydrochloric acid, hydrobromic acid, phosphoric acid, sulfuric acid, or solvates thereof.

5. The pharmaceutical composition of claim 1, wherein the ensifentrine or pharmaceutically acceptable salt or ester thereof is present in a concentration from about 0.001% w/w to about 5% w/w.

6. The pharmaceutical composition of claim 1, wherein the propellant is selected from HFA-152a, HFO-1234ze (E) or a combination thereof.

7. The pharmaceutical composition of claim 6, wherein the propellant HFA-152a, HFO-1234ze (E) or a combination thereof is present in the concentration from about 1% to about 99% w/w of composition.

8. (canceled)

9. The pharmaceutical composition of claim 6, wherein the propellant is a combination of HFA-152a and HFO-1234ze (E).

10. (canceled)

11. The pharmaceutical composition of claim 3, wherein the co-solvent is selected from a group consisting of: ethanol, glycerol, polyethylene glycol and a combination thereof, optionally the co-solvent is present in the concentration from about 0.1% to about 20% w/w of composition.

12. (canceled)

13. The pharmaceutical composition of claim 113, wherein the co-solvent is ethanol and is present in the concentration from about 0.1% to about 20% w/w of the composition.

14. The pharmaceutical composition of claim 3, wherein the surfactant is selected from group consisting of: oleic acid, sorbitan trioleate, sorbitan mono-oleate, polyvinylpyrrolidone, polysorbate and combinations thereof.

15. The pharmaceutical composition of claim 14, wherein the surfactant is present in the concentration from about 0.001% to about 1% w/w of the composition.

16. The pharmaceutical composition of claim 14, wherein the surfactant is polyvinylpyrrolidone, oleic acid, sorbitan trioleate or sorbitan mono-oleate, and present in the concentration from about 0.001% to about 0.005% w/w.

17. (canceled)

18. (canceled)

19. The pharmaceutical composition of claim 3, wherein the lubricant is selected from a group consisting of: silica talc, stearic acid, magnesium or calcium stearate and combinations thereof; optionally the lubricant is present in the concentration from about 0.001% to about 2% w/w of composition.

20. (canceled)

21. The pharmaceutical composition of claim 193, wherein the lubricant is magnesium or calcium stearate and present in the concentration from about 0.001% to about 0.1% w/w of the composition.

22. The pharmaceutical composition of claim 3, wherein the bulking agent is selected from the group consisting of: starches, arabic gums, gelatin, methylcellulose, carboxymethylcellulose, and combinations thereof; optionally wherein the bulking agent is present in the concentration from about 0.01% to about 0.5% w/w of composition.

23. (canceled)

24. The pharmaceutical composition of claim 3, wherein the acid is selected from group consisting of: hydrochloric acid, sulphuric acid, phosphoric acid, citric acid, maleic acid, malic acid, boric acid, methane sulphonic acid, tartaric acid, oxalic acid, and combinations thereof; optionally the acid is present in the concentration from about 0.001 to about 2% w/w of the composition.

25. (canceled)

26. A pharmaceutical suspension composition for treatment or prevention of respiratory disorder in a subject in need thereof, comprising particles of ensifentrine or pharmaceutically acceptable salt or ester thereof, and a green propellant; and wherein the particles of ensifentrine have a particle size distribution with a Dv50 value of from about 1 μm to about 5 μm.

27. The pharmaceutical suspension composition of claim 26, is suitable for administration by oral or nasal inhalation.

28. The pharmaceutical composition of claim 26, wherein formulation can be administered using a pressurized metered dose inhaler or a nebulizer.

29. A pharmaceutical solution composition for treatment or prevention of respiratory disorder in a subject in need thereof, comprising about 0.01% to 2.5% w/w of ensifentrine or pharmaceutically acceptable salt or ester thereof, and HFA-152a or HFO1234ze (E); wherein the ensifentrine is present in the form of extra fine particles having Dv50 of less than 1 μm.
